(19) United States
(12) Patent Application Publication
(10) Pub. No.: US 2022/0340942 A1
(43) Pub. Date: Oct. 27, 2022

(54) SEPARATION OF NEUTRAL OLIGOSACCHARIDES FROM FERMENTATION BROTH

(71) Applicant: Glycom A/S, Hørsholm (DK)

(72) Inventors: Nikolay KHANZHIN, Humlebæk (DK); Pierre CHASSAGNE, Beaumont (FR); Flemming MATHIESEN, Vejle (DK); Jesper THØGERSEN, Varde (DK); Martin MATWIEJUK, Hamburg (DE)

(21) Appl. No.: 17/754,400

(22) PCT Filed: Oct. 1, 2020
(86) PCT No.: PCT/IB2020/059201

§ 371 Date: Mar. 31, 2022

(30) Foreign Application Priority Data
Oct. 1, 2019 (DK) PA 2019 01154

(51) Int. Cl. C12P 19/00; B01D 61/14; B01D 61/16; B01D 61/58; B01J 39/05

(52) U.S. Cl. CPC C12P 19/00; B01D 61/145; B01D 61/16; B01D 61/58; B01J 39/05; B01D 2311/04; B01D 2311/08; B01D 2311/103; B01D 2311/18; B01D 2311/2623; B01D 2311/2649; B01D 2311/2676; B01D 2317/025; B01D 61/027

(57) ABSTRACT

The present invention relates to the separation and isolation of neutral human milk oligosaccharides (HMOs) from the reaction mixture in which they are produced.

### FIELD OF THE INVENTION

The present invention relates to the separation and isolation of neutral human milk oligosaccharides (HMOs) from the reaction mixture in which they are produced.

### BACKGROUND OF THE INVENTION

During the past decades, the interest in the preparation and commercialisation of human milk oligosaccharides (HMOs) has been increasing steadily. The importance of HMOs is directly linked to their unique biological activities, therefore HMOs have become important potential products for nutrition and therapeutic uses. As a result, low cost ways of producing industrially HMOs have been sought.

To date, the structures of more than 140 HMOs have been determined, and considerably more are probably present in human milk (Urashima et al.: Milk oligosaccharides, Nova Biomedical Books, 2011; Chen Adv. Carbohydr. Chem. Biochem. 72, 113 (2015)). The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GlcNAc). Lactose and the N-acetyllactosaminylated or lacto-N-biosylated lactose derivatives may further be substituted with one or more fucose and/or sialic acid residue(s), or lactose may be substituted with an additional galactose, to give HMOs known so far.

Direct fermentative production of HMOs, especially of those being a trisaccharide, has recently become practical (Han et al. Biotechnol. Adv. 30, 1268 (2012) and references cited therein). Such fermentation technology has used a recombinant E. coli system wherein one or more types of glycosyl transferases originating from viruses or bacteria have been co-expressed to glycosylate exogenously added lactose, which has been internalized by the LacY permease of the E. coli. However, the use of a recombinant glycosyl transferase, especially series of recombinant glycosyl transferases to produce oligosaccharides of four or more monosaccharide units, has always led to by-product formation hence resulting in a complex mixture of oligosaccharides in the fermentation broth. Further, a fermentation broth inevitably contains a wide range of non-oligosaccharide substances such as cells, cell fragments, proteins, protein fragments, DNA, DNA fragments, endotoxins, caramelized by-products, minerals, salts or other charged molecules.

For separating HMOs from carbohydrate by-products and other contaminating components, active charcoal treatment combined with gel filtration chromatography has been proposed as a method of choice (WO 01/04341, EP-A-2479263, Dumon et al. Glycoconj. J 18, 465 (2001), Priem et al. Glycobiology 12, 235 (2002), Drouillard et al. Angew. Chem. Int. Ed. 45, 1778 (2006), Gebus et al. Carbohydr. Res. 361, 83 (2012), Baumgartner et al. ChemBioChem 15, 1896 (2014)). Although gel filtration chromatography is a convenient lab scale method, it cannot be efficiently scaled up for industrial production.

Recently, EP-A-2896628 has described a process for purification of 2′-FL from a fermentation broth obtained by microbial fermentation comprising the following steps: ultrafiltration, strong cation exchange resin chromatography (H-form), neutralization, strong anion exchange resin chromatography (acetate-form), neutralization, active charcoal treatment, electrodialysis, second strong cation exchange resin chromatography (H⁺- or Na⁺-form), second strong anion exchange resin chromatography (Cl⁻-form), second active charcoal treatment, optional second electrodialysis and sterile filtration.

WO 2017/182965 and WO 2017/221208 have disclosed a process for purification of LNT or LNnT from fermentation broth comprising ultrafiltration, nanofiltration, active charcoal treatment and treatment with strong cation exchange resin (H⁺-form) followed by weak anion exchange resin (base form).

WO 2015/188834 and WO 2016/095924 have disclosed the crystallization of 2′-FL from a purified fermentation broth, the purification comprising ultrafiltration, nanofiltration, active charcoal treatment and treatment with strong cation exchange resin (H⁺-form) followed by weak anion exchange resin (base form).

Other prior art documents have disclosed purification methods elaborated for low lactose or no-lactose fermentation broths. According to these procedures, lactose added in excess during the fermentative production of a neutral HMO has been hydrolysed in situ after completion of the fermentation by the action of a β-galactosidase, resulting a broth that substantially does not contain residual lactose. Accordingly, WO 2012/112777 has disclosed a series of step to purify 2′-FL comprising centrifugation, capturing the oligosaccharide on charcoal followed by elution and flash chromatography on ion exchange media. WO 2015/106943 has disclosed purification of 2′-FL comprising ultrafiltration, strong cation exchange resin chromatography (H⁺-form), neutralization, strong anion exchange resin chromatography (Cl⁻-form), neutralization, nanofiltration/diafiltration, active charcoal treatment, electrodialysis, optional second strong cation exchange resin chromatography (Na⁺-form), second strong anion exchange resin chromatography (Cl⁻-form), second active charcoal treatment, optional second electrodialysis and sterile filtration. WO 2019/063757 has disclosed a process for purification of a neutral HMO comprising separating biomass from fermentation broth and treatment with a cation exchange material, an anion exchange material and a cation exchange adsorbent resin.

However, alternative procedures for isolating and purifying a neutral HMO from non-carbohydrate components of the fermentation broth in which they have been produced, especially those suitable for industrial scale, are needed to improve the recovery yield of the HMO and/or to simplify prior art methods while the purity of the HMO is at least maintained, preferably improved.

### SUMMARY OF THE INVENTION

The invention relates to a method for obtaining or isolating a neutral human milk oligosaccharide (HMO) from the reaction milieu in which they have been produced, preferably from a fermentation broth, wherein said HMO has been produced by culturing a genetically modified microorganism capable of producing said HMO from an internalized carbohydrate precursor, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C., and
ii) contacting the reaction milieu obtained in step i) to an ultrafiltration (UF) membrane with a molecular weight cut-off (MWCO) of around 5-1000 kDa, wherein the membrane is preferably composed of a non-polymeric material.

Accordingly, in one embodiment, the invention relates to a method for obtaining or isolating a neutral human milk oligosaccharide (HMO) from the reaction milieu in which it has been produced, preferably from a fermentation broth, comprising the steps of

i) optionally, centrifugation, microfiltration of the reaction milieu, or filtration of the reaction milieu on a filter press or a drum filter,
ii) setting the pH of the filtrate or supernatant from step i) or the reaction milieu directly to 3-6 and/or warming up the filtrate or supernatant from step i) or the reaction milieu directly to 35-65° C., and
iii) contacting the reaction milieu obtained in step ii) to an ultrafiltration (UF) membrane with a molecular weight cut-off (MWCO) of 5-1000 kDa, preferably 10-1000 kDa, and collecting the permeate,
with the proviso that when step i) is not carried out, then the UF membrane is a non-polymeric membrane.

Preferably, the method comprises the steps of
i) optionally, centrifugation, microfiltration of the reaction milieu, or filtration of the reaction milieu on a filter press or a drum filter to obtain a pre-treated reaction milieu,
ii) setting the pH of the pre-treated reaction milieu from step i) (which is typically a filtrate or supernatant from step i)) or the reaction milieu directly to 3-6 and optionally warming up the pre-treated reaction milieu from step i) or the reaction milieu directly to 35-65° C., and
iii) contacting the reaction milieu obtained in step ii) to an ultrafiltration (UF) membrane with a molecular weight cut-off (MWCO) of 5-1000 kDa, preferably 10-1000 kDa, and collecting the permeate,
   with the proviso that when step i) is not carried out, then the UF membrane is a non-polymeric membrane.

More preferably, step ii) above comprises both the pH setting and the warming up, particularly the pH setting is made first followed by warming up the so-obtained pH set reaction milieu or pre-treated reaction milieu.

In one embodiment, the method disclosed above further comprises a nanofiltration step.

In other embodiment, the method disclosed above further comprises a treatment with one or more ion exchange resins.

In other embodiment, the method disclosed above further comprises a treatment with or chromatography on active charcoal.

In other embodiment, the method disclosed above further comprise a chromatography using a hydrophobic stationary phase which is a polystyrene cross-linked with divinylbenzene (PS-DVB) and functionalized with bromine on the aromatic ring.

One embodiment of the method relates to obtaining or isolating a neutral HMO from the reaction milieu in which they have been produced, comprising the steps of
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, wherein the membrane is preferably composed of a non-polymeric material, and
iii) contacting the permeate obtained in step ii) with an nanofiltration membrane.

One embodiment of the method relates to obtaining or isolating a neutral HMO from the reaction milieu in which they have been produced, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, wherein the membrane is preferably composed of a non-polymeric material,
iii) contacting the permeate obtained in step ii) with a nanofiltration (NF) membrane, and
iv) treating the retentate obtained in step iii) with an ion exchange resin, preferably a strong cationic ion exchange resin and a weak basic ion exchange resin to demineralize the retentate.

One embodiment of the method relates to obtaining or isolating a neutral HMO from the reaction milieu in which they have been produced, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, wherein the membrane is preferably composed of a non-polymeric material,
iii) contacting the permeate obtained in step ii) with a nanofiltration (NF) membrane, iv) treating the retentate obtained in step iii) with an ion exchange resin, preferably a strong cationic ion exchange resin and a weak basic ion exchange resin to demineralize the retentate, and
v) the solution obtained in step iv) is contacted with activated charcoal (AC) or subjected to chromatography using a hydrophobic stationary phase which is a polystyrene cross-linked with divinylbenzene (PS-DVB) and functionalized with bromine on the aromatic ring, or both in any order.

Preferably, the neutral HMO is 2′-FL, 3-FL, DFL, LNT, LNnT or LNFP-I.

Preferably, the reaction milieu in which the neutral HMO has been produced is a fermentation broth. The fermentation broth typically contains, besides the neutral HMO of interest as main compound for the production of which a genetically modified microorganism, preferably an E. coli, has been suitably designed, carbohydrate by-products or contaminants such as carbohydrate intermediates in the biosynthetic pathway of the neutral HMO of interest from lactose, preferable exogenously added lactose, as precursor, and/or those as a result of a deficient, defective or impaired glycosylation during the biosynthetic pathway, and/or those as a result of rearrangement or degradation under the cultivation condition or post-fermentive operations, and/or lactose as unconsumed educt added in excess during the fermentation. Further, the fermentation broth can contain cells, proteins, protein fragments, DNA, caramelized by-products, minerals, salts, organic acids, endotoxins and/or other charged molecules.

Preferably, when the fermentation broth, before UF, is not subjected to centrifugation, microfiltration or filtration on a filter press or a drum filter, the UF membrane is composed of a non-polymeric material (such as the UF membrane is a ceramic membrane).

Certain embodiments of the invention comprise one or more further optional steps. Preferably, the further optional step is not electrodialysis.

In one embodiment, preferably when the UF permeate is poor in or essentially lacks lactose, the NF step according to step iii) comprises the use of a nanofiltration membrane that has a MWCO that ensures the retention of the neutral human milk oligosaccharide of interest, that is its MWCO is around 25-50% of the molecular weight of the neutral human milk oligosaccharide, typically around 150-500 Da. In this regard the neutral human milk oligosaccharide is accumulated in the NF retentate (NFR), whereas salts such as monovalent ions or monosaccharides are accumulated in the permeate.

In other embodiment, preferably when the UF permeate contains substantial amount of lactose, the NF step according to step iii) comprises the use of a nanofiltration membrane with a MWCO of around 600-3500 Da ensuring the retention of the neutral HMO and allowing mono- and divalent salts and at least a part of the lactose to pass through the membrane, wherein the active (top) layer of the NF membrane is composed of polyamide, and wherein the MgSO₄ rejection factor on the NF membrane is around 50-90%.

In other embodiment, chromatography using a hydrophobic stationary phase which is a polystyrene cross-linked with divinylbenzene (PS-DVB) and functionalized with bromine on the aromatic ring according to step v) is preferably utilized when the neutral HMO of interest is LNT, LNnT or LNFP-I, in order to separate it from contaminating oligosaccharides.

The invention relates, in another aspect, to the separation of a neutral HMO from dissolved inorganic and organic salts, acids and bases in an aqueous medium from a fermentation or enzymatic process, comprising the step of subjecting the aqueous medium to steps i), ii), iii), iv) and optionally step v).

### DETAILED DESCRIPTION OF THE INVENTION

### 1. Terms and Definitions

The term “neutral human milk oligosaccharide” means a non-sialylated (therefore neutral) complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that is a) substituted with one or two α-L-fucopyranosyl moieties, b) substituted with a galactosyl residue, or c) elongated, via its 3′—OH group, by an N-acetylglucosamine, a lacto-N-biose (Galβ1-3GlcNAc) or an N-acetyllactosamine (Galβ1-4GlcNAc) moiety. The N-acetyllactosamine containing derivatives can be further substituted with N-actyllactosamine and/or lacto-N-biose (lacto-N-biose is always a non-reducing terminal). The N-acetyllactosamine and the lacto-N-biose containing derivatives can optionally be substituted by one or more α-L-fucopyranosyl moieties. Examples of neutral trisaccharide HMOs include 2′-O-fucosyllactose (2′-FL, Fucα1-2Gal 1-4Glc), 3-O-fucosyllactose (3-FL, Galβ1-4(Fucα1-3)Glc) or lacto-N-triose II (GlcNAcβ1-3Gal 1-4Glc); examples of neutral tetrasaccharide HMOs include 2′,3-di-O-fucosyllactose (DFL, Fucα1-2Galβ1-4(Fucα1-3)Glc), lacto-N-tetraose (LNT, Galβ1-3GlcNAcβ1-3Galβ1-4Glc) or lacto-N-neotetraose (LNnT, Galβ1-4GlcNAcβ1-3Galβ1-4Glc); examples of neutral pentasaccharide HMOs include lacto-N-fucopentaose I (LNFP I, Fucα1-2Galβ1-3GlcNAcβ1-3Galβ1-4Glc), lacto-N-fucopentaose II (LNFP II, Galβ1-3(Fucα1-4)GlcNAcβ1-3Galβ1-4Glc), lacto-N-fucopentaose III (LNFP III, Galβ1-4(Fucα1-3)GlcNAcβ1-3Galβ1-4Glc), lacto-N-fucopentaose V (LNFP V, Galβ1-3GlcNAcβ1-3Galβ1-4(Fucα1-3)Glc), lacto-N-fucopentaose VI (LNFP VI, Galβ1-4GlcNAcβ1-3Galβ1-4(Fucα1-3)Glc); examples of neutral hexasaccharide HMOs include lacto-N-difucohexaose I (LNDFH I, Fucα1-2Galβ1-3(Fucα1-4)GcNAcβ1-3Gal 1-4Glc), lacto-N-difucohexaose II (LNDFH II, Galβ1-3(Fucα1-4)GlcNAcβ1-3Galβ1-4(Fucα1-3)Glc), lacto-N-difucohexaose III (LNDFH III, Galβ1-4(Fucα1-3)GlcNAcβ1-3Galβ1-4(Fucα1-3)Glc), lacto-N-hexaose (LNH, Galβ1-3GlcNAcβ1-3(Galβ1-4GlcNAcβ1-6)Galβ1-4Glc), para-lacto-N-hexaose (pLNH, Galβ1-3GlcNAcβ1-3Galβ1-4GlcNAcβ1-3Galβ1-4Glc), lacto-N-neohexaose (LNnH, Galβ1-4GlcNAcβ1-3(Galβ1-4GlcNAcβ1-6)Gal 1-4Glc) or para-lacto-N-neohexaose (pLNnH, Galβ1-4GlcNAcβ1-3Galβ1-4GlcNAcβ1-3Galβ1-4Glc).

The term “genetically modified cell” or “genetically modified microorganism” preferably means a cell of a microorganism, such as a bacterial or fungi cell, e.g. an E. coli cell, which has been genetically manipulated to include at least one alteration in its DNA sequence. The term “at least one genetic alteration” means a genetic alteration that can result in a change in the original characteristics of the wild type cell, e.g. the modified cell is able to perform additional chemical transformation due to the introduced new genetic material that encodes the expression of an enzymes not being in the wild type cell, or is not able to carry out transformation like degradation due to removal of gene/genes (knockout). A genetically modified cell can be produced in a conventional manner by genetic engineering techniques that are well-known to those skilled in the art.

The term “genetically modified microorganism capable of producing a neutral HMO from an internalized carbohydrate precursor” preferably means a cell of a microorganism, such as a bacterium or fungi (e.g. yeast), preferably a bacterium, more preferably an E. coli, which is genetically manipulated (vide supra) to comprise one or more endogenous or recombinant genes encoding one or more glycosyl transferase enzymes that are able to transfer the glycosyl residue of an activated sugar nucleotide to an internalized acceptor molecule and necessary for the synthesis of said neutral HMO, a biosynthetic pathway to produce the corresponding activated sugar nucleotide donor(s) suitable to be transferred by said glycosyl transferase to a carbohydrate precursor (acceptor) and a mechanism of internalization of a carbohydrate precursor (acceptor) from the culture medium into the cell where it is glycosylated to produce the neutral HMO of interest. The glycosyl transferases are selected from β-1,3-N-acetylglucosaminyl transferase, β-1,6-N-acetylglucosaminyl transferase, β-1,3-galactosyl transferase, β-1,4-galactosyl transferase, α-1,2-fucosyl transferase, α-1,3-fucosyl transferase and α-1,4 fucosyl transferase. The corresponding activated sugar nucleotides are UDP-Gal, UDP-GlcNAc and GDP-Fuc.

The term “biomass”, in the context of fermentation, refers to the suspended, precipitated or insoluble materials originating from fermentation cells, like intact cells, disrupted cells, cell fragments, proteins, protein fragments, polysaccharides. The term “biomass”, in the context of enzymatic reaction, refers to (mainly denatured and/or precipitated) proteins or protein fragments originating from the enzyme used. The biomass can be separated from the supernatant or the reaction mixture by e.g. centrifugation, microfiltration, ultrafiltration or filtration on a filter press or a drum filter.

The term “Brix” refers to degrees Brix, that is the sugar content of an aqueous solution (g of sugar in 100 g of solution). In this regard, Brix of the N-acetylglucosamine containing neutral oligosaccharide solution of this application refers to the overall carbohydrate content of the solution including the N-acetylglucosamine containing neutral oligosaccharide and its accompanying carbohydrates. Brix is measured by a calibrated refractometer.

Rejection factor of a salt (in percent) is calculated as (1-κₚ/κᵣ) 100, wherein κₚ is the conductivity of the salt in the permeate and κᵣ is the conductivity of the salt in the retentate. The retentate concentration is practically equal to the feed concentration concerning the salt. The procedure for measuring rejection of salts is disclosed in the working examples below.

Rejection factor of a carbohydrate (in percent) is calculated as (1-Cₚ/Cᵣ) 100, wherein Cₚ is the concentration of the carbohydrate in the permeate and Cᵣ is the concentration of the carbohydrate in the retentate. The retentate concentration is practically equal to the feed concentration concerning the carbohydrate. One exemplary procedure for measuring rejection of a carbohydrate is disclosed in the working examples below.

Separation factor concerning two carbohydrates is calculated as (Cₚ₁/Cᵣ₁)/(Cₚ₂/Cᵣ₂), wherein Cₚ₁ and Cₚ₂ are the concentrations of the first and the second carbohydrate, respectively, in the permeate, and Cᵣ₁ and Cᵣ₂ are the concentrations of the first and the second carbohydrate, respectively, in the retentate.

“Pure water flux” is defined as the volume of purified water (e.g. distilled water, RO water) that passes through a membrane per unit time, per unit area under specified conditions (at around 23-25° C., 10 bar and constant cross-flow of 300 l/h).

“Demineralization” preferably means a process of removing minerals or mineral salts from a liquid. In the context of the present invention, demineralization preferably refers to the step of ion exchange treatment, especially a subsequent application of a cation and an anion exchange resin so that the eluate from the second ion exchanger contains no or very low amount of minerals or minerals salts. Moreover, demineralization can occur in the nanofiltration step, especially when it is combined with diafiltration.

“Microfiltration” preferably means a pre-treatment separation processes to filter a fermentation broth or an enzymatic reaction mixture through a membrane having pore ranging from about 0.1 to 10 μm. In terms of approximate molecular weight, these membranes can separate macromolecules of molecular weights generally more than 500,000 g/mol in the retentate.

The terms “around” or “about” used throughout the specification of the invention in connection with a numerical value mean that said numerical value may deviate up to 10% of the indicated value.

### 2. Method for Obtaining or Isolating a Neutral HMO from the Reaction Milieu in which it has been Produced

The invention relates to a method for obtaining or isolation a neutral HMO from an aqueous medium, the aqueous medium being a fermentation broth or an enzymatic reaction mixture in which said neutral HMO has been produced. The reaction milieu is a complex matrix in which the neutral HMO is accompanied or contaminated by several substances like by-product and residual materials necessary for the synthesis of said neutral HMOs. Accordingly, the neutral HMO is obtained or isolated from the reaction milieu by separating it from the by-products and residual materials with the aid of several consecutive steps resulting in that the neutral HMO is obtained or isolated in much purer form than it was in the reaction milieu. The present invention thus provides a purification method by which the neutral HMO of interest can be obtained or isolated in a more beneficial way compared to the prior art. Such benefits are disclosed below with respect to the corresponding method steps.

Using a biotechnological method, whatever the way (fermentation or in vitro enzymatic) the neutral HMO is produced, the reaction milieu contains a biomass. Therefore, the method of invention compulsorily comprises a step of separating the biomass from the reaction milieu to provide an aqueous solution comprising the neutral HMO of interest. Separating the biomass from the reaction milieu comprises ultrafiltration (UF, see details below) optionally preceded by pre-filtration (that is centrifugation, microfiltration, or filtration on a filter press or a drum filter). The UF is usually followed by a nanofiltration step (NF, see details below). Further, the method of invention can optionally, but preferably, comprise a treatment with ion exchange resins, advantageously with a cation and an anion exchange resin. In addition, the method of invention can optionally comprise active charcoal treatment for decolorizing and/or a chromatography step on a neutral solid phase, preferably reversed-phase chromatography, to remove residual hydrophobic contaminants. Any of the optional steps can be performed at any order after UF and NF. Furthermore, the method of invention can optionally comprise at least one more NF step, especially for concentrating and or desalinating/demineralizing the aqueous solution of the neutral HMO. Alternatively, if demineralization is not necessary (e.g. due to low salt content of the feed), the optional additional NF may be replaced by evaporation.

The steps of UF, NF, “treatment with ion exchange resins”, “active charcoal treatment” and “chromatography on a neutral solid phase” are discussed in detail below in the corresponding sub-chapters.

Accordingly, the method comprises the following separation/purification steps in any order:
a) ultrafiltration (UF),
b) nanofiltration (NF), and
c) treatment with an ion exchange resin, and/or chromatography on a neutral solid phase.

Preferably, the method does not comprise electrodialysis.

Advantageously, step a) is conducted before step b). More advantageously, the step a) is conducted before any of the steps b) and c). Preferably, the method is performed in the order where step b) follows step a) and step c) follows step b).

In one embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with ion exchange resins and collecting the resin eluate (RE), and
chromatography of the RE.

In another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
chromatography of the NFR and collecting the chromatography eluate (CE), and
treatment of the CE with ion exchange resins.

The method of the invention may comprise an active charcoal treatment after UF, NF, chromatography or ion exchange resin treatment.

In one embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
treatment of the CCE with ion exchange resins.

Preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
treatment of the CCE with a strong cation exchange resin in H⁺-form and a weak anion exchange resin in free base form.

More preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
treatment of the CCE with a strong cation exchange resin in H⁺-form and a weak anion exchange resin in free base form;
   and the method does not comprise electrodialysis.

In another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with ion exchange resins and collecting the resin eluate (RE), and
activated charcoal treatment of the RE.

Preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with a strong cation exchange resin in H⁺-form and a weak anion exchange resin in free base form, and collecting the resin eluate (RE), and
activated charcoal treatment of the RE.

More preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with a strong cation exchange resin in H⁺-form and a weak anion exchange resin in free base form, and collecting the resin eluate (RE), and
activated charcoal treatment of the RE;
   and the method does not comprise electrodialysis.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
chromatography of the NFR and collecting the chromatography eluate (CE), and
activated charcoal treatment of the CE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
chromatography of the CCE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
chromatography of the NFR and collecting the chromatography eluate (CE),
activated charcoal treatment of the CE and collecting the charcoal eluate (CCE), and
treatment of the CCE with ion exchange resins.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
chromatography of the NFR and collecting the chromatography eluate (CE),
treatment of the CE with ion exchange resins and collecting the resin eluate (RE), and
activated charcoal treatment of the RE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with ion exchange resins and collecting the resin eluate (RE),
chromatography of the RE and collecting the chromatography eluate (CE), and
activated charcoal treatment of the CE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with ion exchange resins and collecting the resin eluate (RE)
activated charcoal treatment of the RE and collecting the charcoal eluate (CCE), and
chromatography of the CCE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE),
treatment of the CCE with ion exchange resins and collecting the resin eluate (RE), and
chromatography of the RE.

Yet in another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE),
chromatography of the CCE and collecting the chromatography eluate (CE), and
treatment of the CE with ion exchange resins.

The method of the invention provides a solution highly enriched with the neutral HMO of interest from which that HMO can be obtained in high yield and preferably with a satisfactory purity, such as that meets the strict regulatory requirements for food applications.

2.1. Production of the Neutral HMO

2.1.1 Production of the Neutral HMO by a Genetically Modified Microorganism

The production of the neutral HMO by culturing a genetically modified cell is preferably performed as the following.

An exogenously added acceptor is internalized from the culture medium by a genetically modified cell where it is converted to the neutral HMO of interest in a reaction comprising one or more enzymatic glycosylation steps. In one embodiment, the internalization can take place via a passive transport mechanism during which the exogenous acceptor diffuses passively across the plasma membrane of the cell. The flow is directed by the concentration difference in the extra- and intracellular space with respect to the acceptor molecule to be internalized, which acceptor is supposed to pass from the place of higher concentration to the zone of lower concentration tending towards equilibrium. In another embodiment, the exogenous acceptor can be internalized in the cell with the aid of an active transport mechanism, during which the exogenous acceptor diffuses across the plasma membrane of the cell under the influence of a transporter protein or permease of the cell. Lactose permease (LacY) has specificity towards mono- or disaccharides such as galactose, N-acetyl-glucosamine, a galactosylated monosaccharide (such as lactose) and an N-acetyl-glucosaminylated monosaccharide. All these carbohydrate derivatives can be easily taken up by a cell expressing a LacY permease (such a cell is also referred herein to as a LacY⁺ phenotype cell) by means of an active transport and accumulate in the cell before being glycosylated (see. e.g. WO 01/04341, Fort et al. J. Chem. Soc., Chem. Comm. 2558 (2005), Drouillard et al. Angew. Chem. Int. Ed. 45, 1778 (2006), WO 2012/112777, WO 2015/036138). Preferably, the cell expressing a lacY gene encoding the lactose permease lacks enzymes that are able to degrade the internalized acceptor and/or the corresponding intermediates during the biosynthetic pathway to the neutral HMO of interest. Preferably, the cell lacks β1,4-galactosidase activity due to the deactivation or deletion of the endogenous lacZ gene (such a cell is also referred herein to as a LacZ-phenotype cell) or at least has a reduced activity of β1,4-galactosidase, see e.g. E. coli with low galactosidase activity according to WO 2012/112777.

In one preferred embodiment, the exogenously added acceptor is lactose, and its internalization takes place via an active transport mechanism mediated by a lactose permease of the cell, more preferably LacY. In other embodiment, the exogenous acceptor is glucose, such as disclosed e.g. in WO 2015/150328.

Being internalized into the cell, the acceptor is glycosylated by means of one or more glycosyl transferases expressed by corresponding heterologous gene(s) or nucleic acid sequence(s) which is/are introduced into the cell by known techniques, e.g. by integrating it/them into the chromosome of the cell or using an expression vector. The glycosyl transferases necessary for making the neutral HMO of interest are selected from β-1,3-N-acetylglucosaminyl transferase, β-1,6-N-acetylglucosaminyl transferase, β-1,3-galactosyl transferase, β-1,4-galactosyl transferase, α-1,2-fucosyl transferase, α-1,3-fucosyl transferase and α-1,4 fucosyl transferase. The genetically modified cell normally comprises a biosynthetic pathway to produce the one or more monosaccharide nucleotide donors suitable to be transferred by the corresponding glycosyl transferase(s). Most of the microorganisms are able to produce UDP-Gal or UDP-GlcNAc via their natural central carbon metabolism. With regard to GDP-Fuc, the genetically modified cell can produced it by two ways. GDP-Fuc can be made by the cell under the action of enzymes involved in the de novo biosynthetic pathway of GDP-Fuc (ManB, ManC, Gmd and WcaG) in a stepwise reaction sequence starting from a simple carbon source like glycerol, fructose or glucose. Alternatively, the genetically modified cell can utilize salvaged fucose that is phosphorylated by a kinase followed by the conversion to GDP-Fuc by a pyrophosphorylase (see e.g. WO 2010/070104).

A neutral HMO can be produced by a genetically modified microorganism in accordance with e.g. Dumon et al. Glycoconj. J 18, 465 (2001), Priem et al. Glycobiology 12, 235 (2002), Dumon et al. Biotechnol. Prog. 20, 412 (2004), Drouillard et al. Angew. Chem. Int. Ed. 45, 1778 (2006), Gebus et al. Carbohydr. Res. 361, 83 (2012), Baumgartner et al. ChemBioChem 15, 1896 (2014) and Enzyme Microb. Technol. 75-76, 37 (2015), WO 01/04341, WO 2010/070104, WO 2010/142305, WO 2012/112777, WO 2014/153253, WO 2015/032412, WO 2015/036138, WO 2015/150328, WO 2015/197082, WO 2016/008602, WO 2016/040531, WO 2017/042382, WO 2017/101958, WO 2017/188684, US 2017/0152538, WO 2018/077892, WO 2018/194411 or WO 2019/008133.

In preferred embodiments, the genetically modified microorganism is E. coli.

Accordingly, in a preferred embodiment, the production process comprises the following steps:
a) providing a genetically modified E. coli cell of LacY⁺ phenotype or LacZ⁺, LacY⁺ phenotype, wherein said cell comprises:
   one or more recombinant genes encoding a glycosyl transferase selected from the group consisting of β-1,3-N-acetylglucosaminyl transferase, β-1,6-N-acetylglucosaminyl transferase, β-1,3-galactosyl transferase, β-1,4-galactosyl transferase, α-1,2-fucosyl transferase, α-1,3-fucosyl transferase and α-1,4 fucosyl transferase necessary for the synthesis of the neutral HMO, and
   one or more genes encoding a biosynthetic pathway to UDP-GlcNAc, UDP-Gal or GDP-Fuc for the corresponding glycosyl transferase listed above, and
b) culturing the genetically modified E. coli cell of LacY⁺ phenotype or LacZ⁻, LacY⁺ phenotype in the presence of exogenous lactose and a suitable carbon source, thereby producing a fermentation broth comprising the neutral HMO.

The fermentation broth so-produced comprises the neutral HMO both in the producing cells and the culture medium. To harvest the intracellular neutral HMO and thereby to raise the titre of the product, the method described above may further comprise an optional step c) of disrupting or permeabilizing the cells, e.g. by heating.

The fermentation broth that comprises the neutral HMO can be accompanied by other carbohydrate compounds. Typically, another carbohydrate compound is lactose which is used as acceptor in the fermentation process for making the neutral HMO and left unconverted. In addition, another accompanying carbohydrate compound can be an intermediary carbohydrate during the biosynthetic pathway to the desired neutral HMO, e.g. lacto-N-triose II in case of producing LNT or LNnT. Although their amounts can be substantially reduced in the fermentation broth before subjecting it to the separation/purification steps disclosed below, e.g. as disclosed in WO 2012/112777 or WO 2015/036138, it is not necessary to do so. The claimed method, in one embodiment, is suitable to separate a neutral HMO accompanied by carbohydrate compounds from non-carbohydrate contaminants, while the relative proportion of the carbohydrate compounds does not substantially change in the course of the claimed method. Therefore the purpose of the claimed method, in one aspect, is a separation of a neutral HMO accompanied by carbohydrate compounds from non-carbohydrate contaminants in an aqueous medium from fermentation broth or enzymatic reaction milieu rather than the purification of the neutral HMO from any other contaminants including accompanying carbohydrate compounds. Neutral human milk oligosaccharides are intended to be used for nutritional purposes, therefore the presence of accompanying carbohydrates besides the main neutral HMO in the final nutritional composition is not adverse, or it can even be advantageous. Another embodiment of the claimed method, however, is suitable to purify the neutral HMO by separating it from carbohydrate and non-carbohydrate contaminations, thereby providing the neutral HMO in substantially pure form.

Accordingly, in one embodiment, wherein the neutral human milk oligosaccharide is LNT and produced by fermentation, the accompanying carbohydrates are mainly lactose (as acceptor employed in the fermentation and left unreacted), lacto-N-triose II (GlcNAcβ1-3Galβ1-4Glc, as intermediary carbohydrate in the biosynthetic pathway to LNT) and p-LNH II (Galβ1-3GlcNAcβ1-3Galβ1-3GlcNAcβ1-3Galβ1-4Glc, as overglycosylated LNT that has similar biological properties to LNT). In another embodiment, wherein the neutral human milk oligosaccharide is LNnT and produced by fermentation, the accompanying carbohydrates are mainly lactose (as acceptor employed in the fermentation and left unreacted), lacto-N-triose II (GlcNAcβ1-3Galβ1-4Glc, as intermediary carbohydrate in the biosynthetic pathway to LNnT) and p-LNnH (Galβ1-4GlcNAcβ1-3Galβ1-4GlcNAcβ1-3Gal 1-4Glc, as overglycosylated LNnT that has similar biological properties to LNnT). In another embodiment, wherein the neutral human milk oligosaccharide is lacto-N-triose II (GlcNAcβ1-3Gal 1-4Glc) and produced by fermentation, the accompanying carbohydrate is mainly lactose (as acceptor employed in the fermentation and left unreacted). In other embodiment, wherein the neutral HMO is 2′-FL (Fucα1-2Galβ1-4Glc) and produced by fermentation, the accompanying carbohydrate is mainly lactose (as acceptor employed in the fermentation and left unreacted) and DFL (Fucα1-2Galβ1-4[Fucα1-3]Glc) as overfucosylated 2′-FL that has similar biological properties to 2′-FL. In other embodiment, wherein the neutral human milk oligosaccharide is LNFP I (a fucosylated LNT, Fucα1-2Galβ1-3GlcNAcβ1-3Galβ1-4Glc) and produced by fermentation, the accompanying carbohydrates are mainly lactose (as acceptor employed in the fermentation and left unreacted), lacto-N-triose II (GlcNAcβ1-3Gal 1-4Glc, as intermediary carbohydrate in the biosynthetic pathway to LNFP-I), LNT (as intermediary carbohydrate in the biosynthetic pathway to LNFP-I), p-LNH II (Galβ1-3GlcNAcβ1-3Galβ1-3GlcNAcβ1-3Galβ1-4Glc, as overglycosylated LNT that has similar biological properties to LNT) and 2′-FL. All the accompanying carbohydrates mentioned above are considered to be neutral HMOs, too.

Moreover, there can be further non-HMO carbohydrate contaminants in the fermentation broth. These are typically lactulose and its glycosylated derivatives. Lactulose may be formed from lactose by rearrangement when lactose is heat-sterilized before adding it to the fermentation and/or during the fermentation. As lactulose is also internalized by the cell, it can be glycosylated, similar to lactose, in a concurrent biotransformation reaction. However, the amount of lactulose and its glycosylated derivatives does not exceed a couple of tenth weight % of the overall dry solid matter of the broth after biomass separation.

According to the invention, the fermentation broth is further subjected to a procedure of separation/purification of the neutral HMO from other non-carbohydrate compounds and optionally from carbohydrate compounds of the broth which is described below.

2.1.2 Production of the Neutral HMO by Means of Ex Vivo Enzymatic Reaction

Neutral HMOs can be produced enzymatically by addition or consecutive addition of monosaccharides to lactose in a reaction or reaction sequence that takes place outside an organism. Typically, the suitable enzyme(s) can be the corresponding glycosyl transferase(s) or (trans)glycosidase(s) that is/are added to the reaction mixture containing the starting acceptor (usually lactose) and the corresponding donor(s) wherein the pH is suitably set. If the neutral HMO is a tetrasaccharide or bigger, the trisaccharide product in the first enzymatic step serves as acceptor for the subsequent enzymatic step to make the tetrasaccharide, and so on. In case of multienzymatic synthesis, the single steps can be performed in separated vessels consecutively; in certain scenario, the enzymatic cascade reaction can be carried out in one flask.

In a glycosyl transferase mediated enzymatic synthesis, the glycosyl transferases necessary for making the neutral HMO of interest from lactose are selected from β-1,3-N-acetylglucosaminyl transferase, β-1,6-N-acetylglucosaminyl transferase, β-1,3-galactosyl transferase, β-1,4-galactosyl transferase, α-1,2-fucosyl transferase, α-1,3-fucosyl transferase and α-1,4 fucosyl transferase, and the donors are selected from UDP-GlcNAc, UDP-Gal and GDP-Fuc, corresponding to the glycosyl transferase(s) used. Such type of enzymatic approaches to neutral HMOs is disclosed in e.g. WO 98/44145, Albermann et al. Carbohydr. Res. 334, 97 (2001) or Scheppokat et al. Tetrahedron: Asymmetry 14, 2381 (2003).

In (trans)glycosidase mediated enzymatic synthesis of neutral HMOs, the suitable enzymes are selected from the group consisting of α1,2-(trans)fucosidase, α1,3-(trans)fucosidase, α1,4-(trans)fucosidase, β1,3-(trans)lacto-N-biosidase, β1,6-(trans)lacto-N-biosidase, β1,3-(trans)N-acetyllactosaminidase, β1,6-(trans)N-acetyllactosaminidase, β1,3-(trans)galactosidase, β1,4-(trans)galactosidase, β1,3-(trans)N-acetylglucosaminidase and β1,6-(trans)N-acetylglucosaminidase. Transglycosidases differ from glycosidases that their hydrolytic activity is reduced and/or they have more considerable transfer activity of the donor to the acceptor. It is possible to produce directed transglycosidase enzyme mutants wherein the hydrolase activity is effaced in favour of the transglycosidase action, e.g. by altering the amino acid sequence. With regard to the suitable donors, they can be di- or oligosaccharides having the sugar moiety to be transferred by the (trans)glycosidase on the non-reducing terminal (e.g. for a α1,2-(trans)fucosidase 2′-FL; for a β1,4-(trans)galactosidase lactose; for a β1,3-(trans)N-acetyllactosaminidase LNnT, etc.) or sugar moieties activated by a good leaving group on the anomeric positions like azide, fluoro, p-nitrophenyl, etc. Such enzymatic reactions to produce neutral HMOs are extensively taught in e.g. WO 2012/156897, WO 2012/156898, WO 2016/063261, WO 2016/063262, WO 2016/157108, WO 2016/199069 or WO 2016/199071, which are incorporated herein by reference.

According to the invention, the enzymatic reaction mixture is further subjected to a procedure of separation/purification of the neutral HMO from other non-carbohydrate compounds and optionally from carbohydrate compounds which is described below.

2.2 Obtention of the Neutral HMO from the Reaction Milieu in which it has been Produced

2.2.1. Optional Pre-Treatment of the Reaction Milieu Before Ultrafiltration

A fermentation broth typically contains, besides the neutral HMO produced, the biomass of the cells of the used microorganism together with proteins, protein fragments, DNA, DNA fragments, endotoxins, biogenic amines, inorganic salts, unreacted carbohydrate acceptors such as lactose, sugar-like by-products, monosaccharides, colorizing bodies, etc. Optionally, in order to make macromolecules of the fermentation broth or the enzymatic reaction mixture more easily filterable, the reaction milieu is subjected to a pH adjustment to around 2.5-7.5 and/or subjected to heat treatment between 30-75° C. and/or clarified by flocculation/coagulation. Also optionally, the reaction milieu, either treated as disclosed above or not, is centrifuged, microfiltered or filtered on a filter press or a drum filter, thereby at least a part of the biomass or the precipitated/flocculated/denatured enzyme(s) is/are removed.

The term “pre-treated reaction milieu” used in the context of the present invention comprises thus at least one of the above steps.

2.2.2. Ultrafiltration (UF) of the Reaction Milieu or the Pre-Treated Reaction Milieu

The UF step of the claimed method comprises:
a) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C., and
b) contacting the reaction milieu obtained in step i) to an ultrafiltration (UF) membrane with a molecular weight cut-off (MWCO) of around 5-1000 kDa, wherein the membrane is preferably composed of a non-polymeric material.

The ultrafiltration step is to separate the biomass (or the remaining part of the biomass of the pre-treated reaction milieu) and, preferably, also high molecular weight suspended solids from the soluble components of the broth which soluble components pass through the ultrafiltration membrane in the permeate. This UF permeate (UFP) is an aqueous solution containing the produced neutral HMO.

In a preferred embodiment, if the reaction milieu is centrifuged, microfiltered or filtered on a filter press or a drum filter, the UF membrane is composed of a non-polymeric material, more preferably a ceramic material.

In one embodiment, the pH of the reaction milieu or the pre-treated, e.g. centrifuged, reaction milieu is set to acidic, such as from around 3 to around 6, preferably to a value that is not higher than around 5, preferably is not higher than around 4, more preferably to a value around 3-4. Setting the pH as disclosed above is especially advantageous, because it gives rise to a substantial reduction in the amount of dissolved biomolecules such as soluble proteins and DNAs due to a more effective denaturation and precipitation. The lower amount of dissolved biomolecules allows to use a higher MWCO UF membrane in the subsequent step that provides a better flux, a contributing factor to a higher productivity.

In other embodiment, the reaction milieu or the pre-treated, e.g. centrifuged, reaction milieu is heated up to a temperature that is higher than an ambient temperature, i.e. room temperature or reaction milieu temperature, e.g. to a temperature within the range of from around 30 to around 90° C., preferably to about 35-85° C., e.g. to around 35-75° C., more preferably to around 50-75° C., such as around 60-65° C. This heat treatment before UF substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

In one embodiment, step a) above comprises setting the pH of said optionally pre-treated reaction milieu and warming up said optionally pre-treated reaction milieu, preferably the pH setting is followed by the warming up. Preferably, step a) above comprises setting the pH of said optionally pre-treated reaction milieu to not higher than 5 and warming up said optionally pre-treated reaction milieu to around 30-90° C., preferably to about 35-85° C., e.g. to around 35-75° C., more preferably to around 50-75° C., such as around 60-65° C., which particularly reduces protein solubility and thereby protein leakage into the UF permeate in the subsequent UF step.

In step b) of the method, a UF membrane composed of a non-polymeric material, preferably a ceramic membrane, is used. The non-polymeric UF membrane tolerates high temperature if UF is carried out at that temperature. Also, the applicable flux of a non-polymeric membrane, advantageously a ceramic membrane, is usually higher than that of a polymeric UF membrane with identical or similar MWCO; an addition, a non-polymeric membrane, advantageously a ceramic membrane, is less prone to fouling or getting clogged. In industrial application, the regeneration of the UF membrane is an important cost and technical factor. A non-polymeric membrane, advantageously a ceramic membrane, allows to use harsh cleaning-in-place (CIP) conditions including caustic/strong acid treatment at high temperature (not applicable on polymeric membranes), which may be required when a fermentation stream with high suspended solid content is ultrafiltered. Furthermore, a non-polymeric membrane, advantageously a ceramic membrane, has longer life-time due to inertness and abrasion-resistance to solid particles that circulate at high cross-flow.

Any conventional non-polymeric ultrafiltration membrane, advantageously ceramic membrane, can be used having a molecular weight cut-off (MWCO) range between about 5 and about 1000 kDa, such as around 10-1000, 5-250, 5-500, 5-750, 50-250, 50-500, 50-750, 100-250, 100-500, 100-750, 250-500, 250-750, 500-750 kDa, or any other suitable sub-ranges.

Step b) of the method can be conducted at low temperature (around 5° C. to rt), at around room temperature or at elevated temperature, preferably at elevated temperature. The elevated temperature preferably does not exceed around 65° C.; suitable temperature ranges can be e.g. around 35-50, 35-65, 45-65, 50-65, 55-65 or 60-65° C. The UF step conducted at elevated temperature substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

Preferably, if step b) is carried out at elevated temperature, it is advantageous to set the pH of the reaction milieu to not higher than around 5 in the preceding step a), because it particularly reduces protein solubility and thereby protein leakage into the UF permeate.

The ultrafiltration step can be applied in dead-end or cross-flow mode.

In one embodiment, only a single UF step is conducted in the method of the invention.

In other embodiment, the method of the invention may comprise more than one ultrafiltration steps using membranes with different MWCO, e.g. using two ultrafiltration separations wherein the first membrane has a higher MWCO than that of the second membrane, provided that at least one the UF membranes is a non-polymeric membrane, preferably a ceramic membrane. This arrangement may provide a better separation efficacy of the higher molecular weight components of the broth.

Yet in one embodiment, the ultrafiltration can be combined with diafiltration.

After conducting ultrafiltration comprising steps a) and b) disclosed above, the UF permeate contains materials that have a molecular weight lower than the MWCO of the utilized membrane, optionally the MWCO of the last membrane when a series of membranes are used, including neutral HMO of interest.

2.2.3. Nanofiltration

The method of the invention comprises a nanofiltration (NF) step. Preferably, the NF step directly follows the ultrafiltration of the reaction milieu or the centrifuged reaction milieu, that is the feed of the NF step is the UF permeate containing the neutral HMO of interest. Optionally, the UF permeate can be decolorized by using active charcoal (see below) before conducting the NF step. This nanofiltration step may advantageously be used to concentrate UF permeate and/or to remove ions, mainly monovalent ions, and organic materials having a molecular weight lower than that of the neutral HMO, such as monosaccharides. The nanofiltration membrane has a lower MWCO than that of the ultrafiltration membrane(s) used in the precedent step and ensures the retention of the neutral HMO of interest.

In a first aspect of nanofiltration, the MWCO of the NF membrane is around 25-50% of the molecular weight of the neutral HMO of interest, typically around 150-500 Da. In this regard, the neutral HMO of interest is accumulated in the NF retentate (NFR). The nanofiltration can be combined with diafiltration with water to remove, or to reduce the amount of, permeable salts such as monovalent ions more effectively.

In one embodiment of the first aspect, NF follows UF so that the UF permeate is nanofiltered without diafiltration, and the NF retentate containing the neutral HMO is collected and subjected to further separation step(s) of the method.

In other embodiment of the first aspect, NF follows UF so that the UF permeate is nanofiltered followed by diafiltration, and the NF retentate containing the neutral HMO is collected and subjected to further separation step(s) of the method.

The first aspect of the nanofiltration is advantageously applicable for UF permeate that does not contain lactose or contains only minor amount of lactose (at most around 1-2% of the weight of the neutral HMO of interest).

In the second aspect of nanofiltration, which is advantageously applicable for UF permeate that contains more lactose, the membrane has a MWCO of 600-3500 Da ensuring the retention of the tri- or higher neutral HMO and allowing at least a part of lactose to pass through the membrane, wherein the active (top) layer of the membrane is composed of polyamide, and wherein the MgSO₄ rejection factor on said membrane is around 20-90%, preferably 50-90%.

The term “ensuring the retention of the tri- or higher neutral HMO” preferably means that, during the nanofiltration step, the tri- or higher neutral HMOs do not pass, or at least significantly do not pass, through the membrane and thus their vast majority will be present in the retentate. The term “allowing at least a part of lactose to pass through the membrane” preferably means, that lactose, at least partially, can penetrate the membrane and be collected in the permeate. In case of high rejection (about 90%) of lactose, a subsequent diafiltration with pure water may be necessary to bring all or at least the majority of the lactose in the permeate. The higher the lactose rejection the more diafiltration water is necessary for efficient separation.

The applied nanofiltration membrane according to the second aspect of NF shall be tight for tri- and higher neutral HMOs in order that they are efficiently retained. Preferably, the rejection of the tri- or higher neutral HMO is more than 95%, more preferably 97%, even more preferably 99%. Membranes with MWCO of more than 3500 Da are expected to allow more or significant amount of tri- or higher neutral HMOs pass through the membrane thus show a reduced retention of tri- or higher neutral HMO and therefore are not suitable for the purposes of the invention, and can be excluded. It is preferred that the rejection of the lactose is not more than 80-90%. If the lactose rejection turns to be 90±1-2%, the tri- or tetrasaccharide neutral HMO rejection shall preferably be around 99% or higher in order to achieve a practically satisfying separation.

Oligosaccharide rejection factors and separation factors are measured and calculated as disclosed in WO 2019/003133.

The above requirements are simultaneously fulfilled when the membrane is relatively loose for MgSO₄, that is its rejection is about 50-90%. In this regard the above specified membrane is tight for tri- and higher neutral HMOs, and loose for monosaccharides and lactose, and as well as for MgSO₄. Therefore, it is possible to separate lactose, which is a precursor in making human milk oligosaccharides enzymatically or by fermentation, from the neutral human milk oligosaccharides product by nanofiltration with a good efficacy, and additionally a substantial part of divalent ions also passes to the permeate. In some embodiments, the MgSO₄ rejection factor is 30-90%, 20-80%, 40-90%, 40-80%, 60-90%, 70-90%, 50-80%, 50-70%, 60-70% or 70-80%. Preferably, the MgSO₄ rejection factor on said membrane is 80-90%. Also preferably, the membrane has a rejection factor for NaCl that is lower than that for MgSO₄. In one embodiment, the rejection factor for NaCl is not more than around 50%. In other embodiment, the rejection factor for NaCl is not more than around 40%. In other embodiment, the rejection factor for NaCl is not more than around 30%. In other embodiment, the rejection factor for NaCl is not more than around 20%. At a NaCl rejection of around 20-30%, a substantial reduction of all monovalent salts in the retentate is also achievable.

Determination of NaCl and MgSO₄ rejection on a membrane is disclosed in WO 2019/003133.

Also preferably, in some embodiments, the pure water flux of the membrane is at least 50 l/m²h (when measured at 23-25° C., 10 bar and constant cross-flow of 300 l/h). Preferably, the pure water flux of the membrane is at least 60 l/m²h, at least 70 l/m²h, at least 80 l/m²h or at least 90 l/m²h.

The active or the top layer of nanofiltration membrane suitable in the second aspect of the NF step is preferably made of polyamide. Although membranes of different type seem to have promising separation efficacy, for example NTR-7450 having sulphonated PES as active layer for separating lactose and 3′-SL (Luo et al. (Biores. Technol. 166, 9 (2014); Nordvang et al. (Separ. Purif Technol. 138, 77 (2014)), the above specified membrane used in the invention shows always better separation of lactose from a neutral HMO. In addition, the above mentioned NTR-7450 membrane is subject to fouling, which typically results in a drop in flux, increasing the lactose rejection and therefore a reduced separation factor. Yet preferably, the polyamide membrane is a polyamide with phenylene diamine or piperazine building blocks as amine, more preferably piperazine (referred to as piperazine-based polyamide, too).

Yet preferably, the membrane suitable for the purpose of the present invention is a thin-film composite (TFC) membrane.

An example of suitable piperazine based polyamide TFC membranes is TriSep® UA60.

The nanofiltration membrane suitable in the second aspect of the NF step is characterized by some or all of the above features and thus one or more of the following benefits are provided: selectively and efficiently removes lactose, from tri- or higher neutral HMOs yielding an enriched tri- or higher neutral HMO fraction; removes efficiently monovalent as well as divalent salts therefore no ion exchange step may be necessary or, if desalination is still needed, the ion exchange treatment requires substantially less resin; higher flux during the nanofiltration can be maintained compared to other membranes used for the same or similar purpose in the prior art, which reduces the operation time; the membrane disclosed above is less prone to getting clogged compared to the prior art solutions; the membrane disclosed above can be cleaned and regenerated completely therefore can be recycled without substantial reduction of its performance.

In one embodiment of the second aspect of the NF step, the step comprises:
contacting the UF permeate with a nanofiltration membrane with a molecular weight cut-off (MWCO) of 600-3500 Da ensuring the retention of the tri- or higher neutral HMO and allowing at least a part of the lactose to pass through the membrane, wherein the active (top) layer of the membrane is composed of polyamide, and wherein the MgSO₄ rejection factor on said membrane is around 50-90%,
a subsequent optional diafiltration with said membrane,
and collecting the retentate enriched in the tri- or higher neutral HMO.

In other embodiment, the NF step comprises:
contacting the UF permeate with a piperazine-based polyamide nanofiltration membrane with a molecular weight cut-off (MWCO) of 600-3500 Da, preferably 1000-3500 Da, ensuring the retention of the tri- or higher neutral HMO and allowing at least a part of the lactose to pass through the membrane, wherein the MgSO₄ rejection factor on said membrane is around 80-90%, and wherein
   the NaCl rejection factor on said membrane is lower than that for MgSO₄, and/or
   the pure water flux value of said membrane is at least around 50 l/m²h,
a subsequent optional diafiltration with said membrane,
and collecting the retentate enriched in the tri- or higher neutral HMO.

Preferably, the NaCl rejection factor of the membrane is at most the half of the MgSO₄ rejection factor.

To achieve all the benefits mentioned above, the nanofiltration membrane to be applied in the second aspect of the NF step, preferably:
is a piperazine-based polyamide membrane with a MWCO of 600-3500 Da, preferably 1000-3500 Da,
has a MgSO₄ rejection of around 50-90%, preferably 80-90%,
has a NaCl rejection of not more than around 30%, and
has a pure water flux value of at least around 50 l/m²h (measured at 23-25° C., 10 bar and constant cross-flow of 300 l/h), preferably around 90 l/m²h.

In one embodiment of the second aspect of the NF step, the separation factor of the lactose over a tri- or higher neutral HMO is more than around 10, preferably more than 15-25, more preferably more than 30-50, even more preferably more than 75-100.

In other embodiment of the second aspect of the NF step, the separation factor of lactose over LNTri II is more than around 10, preferably more than around 20, more preferably more than around 30.

In other embodiment of the second aspect of the NF step, the separation factor of lactose over LNT or LNnT is more than around 30, more preferably more than around 50.

In other embodiment of the second aspect of the NF step, the separation factor of lactose over pLNnH or pLNH II is more than around 150, more preferably more than around 250.

The second aspect of the NF step can be conducted under conditions used for conventional nanofiltration with tangential flow or cross-flow filtration with positive pressure compared to permeate side followed by diafiltration where both operations could be performed in a batch mode or preferably in continuous mode. The optional diafiltration is conducted by adding pure water to the retentate after the nanofiltration step disclosed above and continuing the filtration process with constant removal of permeate under the same or similar conditions as nanofiltration. The preferred mode of water addition is continuous, i.e. the addition flow rate is matching approximately the permeate flow rate. NF could be performed in a batch mode where retentate stream is recycled back to the feed tank and the diafiltration (DF) is done by adding purified or deionized water to the feed tank continuously. Most preferably, DF water is added after at least some pre-concentration by removing a certain amount of permeate. The higher concentration factor before the start of DF the better DF efficacy is achieved. After completion of DF, further concentration could be achieved by removing extra amount of permeate. Alternatively, NF could be performed in continuous mode preferably in multi-loop system where retentate from each loop is transferred to the next loop. In this case, DF water could be added separately in each loop with the flow rate either matching the permeate flow rate in each loop or at a lower flow rate. Like in batch mode DF, to improve the efficacy of DF, less water or no water should be added in the e.g. first loop to achieve a higher concentration factor. The distribution of water in multi-loop system as well as other process parameters such as trans-membrane pressure, temperature and cross-flow is subject to routine optimization.

The pH of the feed solution applied for second aspect of the NF step is, preferably, not higher than around 7, more preferably between around 3 and 7, even more preferably around 4 and 5, or around 5 and 6. A pH that is lower than 3 may adversely influence the membrane and the solute properties.

The convenient temperature range applied for second aspect of the NF step is between around 10 and around 80° C. Higher temperature provides a higher flux and thus accelerates the process.

The membrane is expected to be more open for flow-through at higher temperatures, however this doesn't change the separation factors significantly. A preferred temperature range for conducting the nanofiltration separation according to the invention is around 15-45° C., such as 20-45° C.

A preferred applied pressure in the nanofiltration separation is about 2-50 bars, such as around 10-40 bars. Generally, the higher the pressure the higher the flux.

In certain embodiments, the method of the invention may comprise additional (one or more) NF steps, preferably following an active charcoal treatment (see below) and/or ion exchange treatment (see below) and/or chromatography on neutral solid phase (see below), wherein the main purpose is to concentrate the aqueous solution containing the neutral HMO of interest.

2.2.4. Treatment with an Ion Exchange Resin

The aqueous solution of the neutral HMO obtained as UF permeate disclosed above or NF retentate disclosed above may optionally be further purified by means of an ion exchange resin. Any the UF permeate and the NF retentate can optionally be decolorized by using active charcoal (see below) before the treatment with ion exchange resin. Residual salts, colour bodies, biomolecules that contains ionizable groups (such as proteins, peptides, DNA and endotoxins), neutral or zwitter-ionic compounds containing ionizable functional groups (such as amino groups in metabolites including biogenic amines, amino acids), compounds with acid-containing groups (such as organic acids, amino acids) can be further removed by treatment with resin. Especially, low salt content of the resin eluate (demineralization) can be achieved if a cation and an anion exchange resin is applied in the “treatment with an ion exchange resin”.

According to one embodiment, the ion exchange resin is a cation exchange resin, preferably a strongly acidic cation exchange resin, preferably in protonated form. In this step, the positively charged materials can be removed from the feed solution as they bind to the resin. The solution of the neutral HMO is contacted with the cation exchange resin in any suitable manner which would allow positively charged materials to be adsorbed onto the cation exchange resin, and the neutral HMO to pass through. The resulting liquid, after contacting with the cation exchange resin, contains the neutral HMO besides anions in the form of the corresponding acids) and neutral carbohydrates like lactose (if still left after one or more previous purification steps). The acids can be neutralized conventionally.

According to one embodiment, the ion exchange resin is an anion exchange resin. The anion exchange resin can be a strong anion exchange resin with preferably OH⁻-form. In this step, the negatively charged materials can be removed from the feed solution as they bind to the resin. The aqueous solution of the neutral HMO is contacted with an anion exchange resin in any suitable manner which would allow the negatively charged materials to be adsorbed onto the anion exchange resin, and the neutral HMO to pass through. The resulting liquid, after contacting with the anion exchange resin, contains primarily water, cations (in the form of the corresponding bases) and neutral carbohydrates like lactose (if still left after one or more previous purification steps). The bases can be neutralized conventionally.

One of the ion exchange resin treatments disclosed above may be sufficient to obtain the neutral HMO in a required purity. If necessary, both cation and anion exchange resin chromatography, in any order, can be applied.

In one embodiment, if both cation and anion resin treatment are applied, the cation exchange resin in H-form and an anion exchange resin is a weak anion resin in free base form. The cation exchange resin is preferably a strong exchanger. This particular arrangement, besides removing salts and charged molecules from the remaining culture medium, can physically adsorb proteins, DNA and colorizing/caramel bodies efficiently that were left in the culture medium.

The application of a weak basic anion exchanger in free base form (that is, wherein the resin's functional group is a primary, secondary or tertiary amine) is advantageous compared to that of a strong basic anion exchanger in OH⁻-form. The strong basic exchanger has the ability, due to its strong basicity, to deprotonate the anomeric OH-group of the neutral HMO. This initiates rearrangement reactions in the structure of the neutral HMO and thus creates by-products, and/or a significant amount of neutral HMO is bound to the resin. Consequently, both events contribute to the reduction of the recovery yield of the neutral HMO.

Moreover, the application of a weak anion exchange resin in free base form directly after a strong cation exchange resin treatment in H-form has additional advantages:
it effectively neutralizes the acidic eluent collected from the cation exchanger therefore providing a neutral eluent without requiring an extra neutralizing step between the two ion exchanger treatments,
it does not introduce anions of the strong anion exchanger like Cl⁻, AcO⁻ or HCO₃⁻ which would need to be removed later,
the present setup efficiently lowers the conductivity of the resin load by almost two orders of magnitude and directly provides an eluent with low conductivity (below around 200 μS/cm, preferably below around 100 μS/cm, more preferably below around 50 μS/cm), consequently with low salt electrolyte content.

In one embodiment, the optional ion exchange resin treatment step of the claimed method consists of the treatment of the aqueous solution of the neutral HMO, which aqueous solution is an UF permeate, which can optionally be decolorized with active charcoal, as disclosed above, or a NF retentate, which can optionally be decolorized with active charcoal, as disclosed above, with a strong cation exchange resin in H⁺-form directly followed by a treatment with a weak anion exchange resin in free base form.

In other embodiment, the claimed method for obtaining or isolating a neutral HMO from the reaction milieu in which they have been produced:
comprises an ion exchange resin treatment step that consists of the treatment of the aqueous solution of the neutral HMO, which aqueous solution is an UF permeate, which can optionally be decolorized with active charcoal, as disclosed above or a NF retentate which can optionally be decolorized with active charcoal, as disclosed above, with a strong cation exchange resin in H⁺-form directly followed by a treatment with a weak anion exchange resin in free base form, and
does not comprise electrodialysis.

In other embodiment, the claimed method for obtaining or isolating a neutral HMO from the reaction milieu in which they have been produced comprises only a single ion exchange resin treatment step that consists of the treatment of the aqueous solution of the neutral HMO, which aqueous solution is an UF permeate, which can optionally be decolorized with active charcoal, as disclosed above, or a NF retentate, which can optionally be decolorized with active charcoal, as disclosed above, with a strong cation exchange resin in H⁺-form directly followed by a treatment with a weak anion exchange resin in free base form. With a single ion exchange treatment step consisting of a strong cation exchange resin in H⁺-form directly followed by a weak anion exchange resin in free base form reduces the conductivity of the eluate as much as the resin set-up of three ion exchangers disclosed in WO 2019/063757 does. In addition, at least around 10-fold colour reduction of the feed solution is achievable if the resin feed is not treated previously with active charcoal.

In other embodiment, the claimed method for obtaining or isolating a neutral HMO from the reaction milieu in which they have been produced does not comprise any ion exchange treatment step, provided that the method comprises crystallization of the neutral HMO, preferably as the final step. Advantageously, the neutral HMO is 2′-FL and the crystallization is conducted using aqueous acetic acid solution, e.g. as disclosed in WO 2016/095924.

When using an ion exchange resin, its degree of crosslinking can be chosen depending on the operating conditions of the ion exchange column. A highly crosslinked resin offers the advantage of durability and a high degree of mechanical integrity, however suffers from a decreased porosity and a drop off in mass-transfer. A low-crosslinked resin is more fragile and tends to swell by absorption of mobile phase. The particle size of the ion exchange resin is selected to allow an efficient flow of the eluent, while the charged materials are still effectively removed. A suitable flow rate may also be obtained by applying a negative pressure to the eluting end of the column or a positive pressure to the loading end of the column, and collecting the eluent. A combination of both positive and negative pressure may also be used. The ion exchange treatment can be carried out in a conventional manner, e.g. batch-wise or continuously.

Non-limiting examples of a suitable acidic cation exchange resin can be e.g. Amberlite IR100, Amberlite IR120, Amberlite FPC22, Dowex 50WX, Finex CS16GC, Finex CS13GC, Finex CS12GC, Finex CS11GC, Lewatit S, Diaion SK, Diaion UBK, Amberjet 1000, Amberjet 1200, Dowex 88.

Non-limiting examples of a suitable basic anion exchange resin can be e.g. Amberlite IRA67, Amberlite IRA 96, Amberlite IRA743, Amberlite FPA53, Diaion CRB03, Diaion WA10, Dowex 66, Dowex Marathon, Lewatit MP64.

For the single ion exchange treatment step consisting of a strong cation exchange resin in H⁺-form directly followed by a weak anion exchange resin in free base form, the amount of the neutral HMO of interest in the resin load, for both resins, is at least around 0.5 kg/l resin, more preferably at least around 0.8 kg/l resin, such as around 1.0-1.5 kg/l resin, wherein the volume of the resin corresponds to the volume of the wet resin, that is fully swollen in water.

2.2.5 Active Charcoal Treatment

According to certain embodiments, the method of invention comprises the optional step of active charcoal treatment. The optional active charcoal treatment may follow any of UF step, NF step or the ion exchange treatment step, all disclosed above. The active charcoal treatment helps to remove colorizing agents and/or to reduce the amount of other water soluble contaminants, such as salts, if required. Moreover, the active charcoal treatment removes residual or trace protein, DNA or endotoxin that may remain incidentally after the previous steps.

A carbohydrate substance like a neutral HMO of interest tends to bind to the surface of charcoal particles from its aqueous solution, e.g. an aqueous solution obtained after the UF step, the NF step or the ion exchange treatment step. Similarly, the colorizing agents are also capable to adsorb to the charcoal. While the carbohydrates and colour giving materials are adsorbed, water soluble materials that are not or are more weakly bound to the charcoal can be eluted with water. By changing the eluent from water to aqueous alcohol, e.g. ethanol, the adsorbed neutral HMO can easily be eluted and collected in a separate fraction. The adsorbed colour giving substances would still remain adsorbed on the charcoal, thus both decolourization and partial desalination can be achieved simultaneously in this optional step. However, due to the presence of organic solvent (ethanol) in the elution solvent, the efficacy of decolorization is lower compared to the case when the elution is done with pure water (see below).

Under certain conditions, the neutral HMO is not, or at least not substantially, adsorbed to the charcoal particles and elution with water gives rise to an aqueous solution of the neutral HMO without a significant loss in its amount, while the colour giving substances remain adsorbed. In this case, there is no need to use organic solvent such as ethanol for elution. It is a matter of routine skills to determine the conditions under which the neutral HMO would bind to the charcoal from its aqueous solution. For example, in one embodiment, a more diluted solution of the neutral HMO or a higher amount of charcoal relative to the amount of the neutral HMO is used, in another embodiment a more concentrated solution of the neutral HMO and a lower amount of charcoal relative to the amount of the neutral HMO is applied.

The charcoal treatment can be conducted by adding charcoal powder to the aqueous solution of the neutral HMO under stirring, filtering off the charcoal, re-suspending in aqueous ethanol under stirring and separating the charcoal by filtration. In higher scale purification, the aqueous solution of neutral HMO is preferably loaded to a column packed with charcoal, which may optionally be mixed with celite, then the column is washed with the required eluent. The fractions containing the neutral HMO are collected. Residual alcohol, if used for elution, may be removed from these fractions by e.g. evaporation, to give an aqueous solution of the neutral HMO.

Preferably, the active charcoal used is granulated. This ensures a convenient flow-rate without applying high pressure.

Also preferably, the active charcoal treatment, more preferably the active charcoal chromatography, of the aqueous solution containing the neutral HMO of interest from the UF step, NF step or the ion exchange treatment step, is conducted at elevated temperature. At elevated temperature, the binding of colour bodies, residual proteins, etc. to the charcoal particles takes place in a shorter contact time, therefore the flow-rate can be conveniently raised. Moreover, the active charcoal treatment conducted at elevated temperature substantially reduces the total number of viable microorganisms (total microbial count) in the aqueous solution of the neutral HMO, thus a sterile filtration step in the later phase of the method may not be necessary. The elevated temperature is at least 30-35° C., such as at least 40° C., at least 50° C., around 40-50° C. or around 60° C.

Also preferably, the amount the applied charcoal is not more than around 10 weight % of the neutral HMO contained in the load, more preferably around 2-6 weight %. This is economical, because all the benefits disclosed above can be conveniently achieved with a very low amount of charcoal.

It is especially preferred when the active charcoal treatment, preferably chromatography, is conducted at elevated temperature with not more than around 10 weight %, preferably around 2-6 weight %, of active charcoal relative to the amount of the neutral HMO of interest in the aqueous solution.

2.2.6 Chromatographic Purification on a Neutral Solid Phase

In this optional step, the aqueous solution of the neutral HMO from UF step, NF step, the ion exchange treatment step or active charcoal treatment, all disclosed above in detail, may be further purified by means of chromatography on a neutral solid phase.

The resulting aqueous solution after UF step, NF step, the ion exchange treatment step or active charcoal treatment may contain small amounts of other soluble hydrophobic impurities which should be removed. The impurities may be removed by subjecting this aqueous medium to a chromatography on a neutral solid phase, advantageously a reversed-phase chromatography. Thereby, contaminants that contain a hydrophobic moiety are adsorbed and consequently retained, due to hydrophobic interactions, with the hydrophobic ligands, such as alkyl or aryl side chains, of the gel matrix (resin) of the stationary phase, while the more hydrophilic neutral HMO does not bind onto the reversed-phase chromatographic medium and therefore is eluted with the aqueous medium, used as the mobile phase.

The reversed-phase chromatography can be carried out in a conventional manner. Preferably, a hydrophobic chromatographic medium is used that is selected from the group consisting of: reversed-phase silicas and organic polymers, especially copolymers of styrene or divinylbenzene and methacrylate polymer. The silicas are preferably derivatized with straight chain alkyl hydrocarbons ranging in length from C1 to C18 (C1, C4, C5 C8 and C18 being the most common) or other hydrophobic ligands (for example phenyl or cyano).

To the aqueous medium used as the mobile phase in the reversed-phase chromatography an organic solvent may be added to alter its polarity, thereby to enhance purification. Many organic solvents, preferably solvents miscible with water, can be used for this purpose, like lower alkanols, such as methanol, ethanol and isopropanol, or acetonitrile, or tetrahydrofuran, or acetone.

Also to enhance the purification of the neutral HMO, the pH of the aqueous medium is preferably adjusted to pH around 3-8, such as around 4-7, prior to reversed-phase chromatography. This is preferably accomplished in a conventional manner by addition, for example, of ammonium formate, ammonium acetate or ammonium hydrogen carbonate.

Also to enhance the purification of the neutral HMO, a salt is preferably dissolved in the aqueous medium prior to reversed-phase chromatography. The salt increases the hydrophobic interactions of the non-saccharide contaminants to increase their removal by the hydrophobic chromatographic medium. Salts that can be used include: Na₂SO₄, K₂SO₄, (NH₄)₂SO₄, NaCl, NH₄Cl, NaBr, NaSCN and NaClO₄.

The reversed-phase chromatography can otherwise be carried out in a conventional manner, e.g. batch-wise or continuously. The purification can be easily done by using a conventional chromatographic column or container of laboratory or industrial scale, in which the hydrophobic chromatographic medium can be either packed or suspended (e.g. as beads).

2.2.6.1 Chromatography on Bromine Functionalized PS-DVB Hydrophobic Stationary Phase

Typically, the application of highly hydrophobic stationary chromatographic medium is not suitable for separating highly polar oligosaccharides (see e.g. WO 2017/221208). However, during the microbial or enzymatic production of the neutral HMO of interest, accompanying oligosaccharides other than the neutral HMO of interest, as by-products, may be formed (see 2.1 above). Some of them may be removed directly from, or at least their amounts may be reduced in, the reaction milieu (see 2.1 above) or with nanofiltration under special conditions (see 2.2.2 above).

If the neutral HMO of interest is required to be available in high purity with very low amount of accompanying oligosaccharide contamination, a further purification step comprising a chromatography on bromine functionalized polystyrene cross-linked with divinylbenzene (PS-DVB) hydrophobic stationary phase may be necessary.

Preferably, the level of bromination of the BPS-DVB resin is about 25-61 w/w %, for example about 25-35 w/w %.

The above chromatographic separation process is robust both as a batch process and in multi-column set-up, spanning from R&D lab through pilot plant to industrial full-scale. The solid phase and the associated chromatographic run can be done in a fashion where a gradient is applied using e.g. aqueous alcohol, but can also be run completely without organic solvents (pure water). The process well-functions at high temperature (e.g. up to around 60° C.) which provides a benefit in terms of reduced risk of microbial growth and increased productivity. In addition, the solid phase can be fully regenerated using e.g. aqueous acetic acid and thereby very suitable for food-related processing.

The separation method can be carried out in a conventional manner. The aqueous solution comprising the neutral HMO of interest used as the mobile phase in the chromatography. An organic solvent, preferably a C1-C4 alcohol, may be added to the aqueous solution. The pH of the aqueous solution is preferably between around 3 to around 8, more preferably between around 4 to around 7. If necessary, the pH can be adjusted to the required value in a conventional manner by addition of an aqueous solution of an acid, a base or a buffer. The separation can be easily done by using a conventional chromatographic column or container of laboratory or industrial scale, in which the PS-DVB-Br resin can be either packed or suspended (e.g. as beads). Preferably, separation method is performed in a column.

The degree of separation depends on many parameters, such as the nature of the flow/elution rate, volumes of fractions collected, mass of the oligosaccharide load relative to the resin mass or resin bed volume, etc. These parameters can be optimized with routine skills. The term “separation” means the full separation of the neutral HMO of interest from the accompanying oligosaccharide(s), that is it is collected and isolated from the fractions in pure form not containing the other oligosaccharide(s). Also, the term “separation” means a partial separation wherein the neutral HMO of interest can be obtained from at least one fraction in pure form or the ratio of the neutral HMO of interest to the accompanying oligosaccharide(s) in the fraction(s) is higher than that in the feed solution, thereby the neutral HMO of interest is enriched.

Preferably, the chromatography on the BPS-DVB stationary medium comprises:
loading the aqueous solution containing the neutral HMO of interest onto the BPS-DVB medium,
eluting with water optionally containing a C₁-C₄ alcohol, and then
collecting the fractions at least enriched in the neutral HMO of interest.

After chromatography, the BPS-DVB medium can be regenerated by elution with water containing water-miscible organic solvents and recycled.

In the method of separating the neutral HMO of interest from an accompanying oligosaccharide by chromatography on BPS-DVB described above the neutral HMO of interest and the accompanying oligosaccharide differ from each other in at least one structural feature, e.g. at least one monosaccharide unit is different, the number of monosaccharide units is different or the orientation of at least one of the interglycosidic linkages is different (whether α or β). In one embodiment, one of the oligosaccharides consists of at least two monosaccharides units more than the other, for example one of the oligosaccharides is a trisaccharide and the other is a pentasaccharide, or one of the oligosaccharides is a tetrasaccharide and the other is a hexasaccharide. In other embodiment, one of the neutral HMO of interest comprises at least one GlcNAc-unit, whereas the accompanying oligosaccharide does not. In other embodiment, one of the oligosaccharides comprises more GlcNAc-units than the other.

Preferably, the neutral HMO of interest is a tetrasaccharide, e.g. LNnT, and the accompanying oligosaccharide is a penta- or hexasaccharide, e.g. pLNnH. Also preferably, the neutral HMO tetrasaccharide is LNT, and the accompanying oligosaccharide is a penta- or hexasaccharide, e.g. pLNH II.

2.2.7 Providing the Neutral HMO of Interest in Isolated Form

After the separation/purification steps disclosed in any of 2.2.2 to 2.2.6 above, the neutral HMO of interest so-obtained can be provided in its solid form by means of spray-drying, freeze-drying or crystallization. Accordingly, the method of the invention may comprise one or more further steps of providing the neutral HMO of interest in isolated, preferably, dried form, such as a step of spray-drying an aqueous solution of the neutral HMO obtained after the UF step, NF step, active charcoal treatment, ion exchange treatment and/or chromatography on neutral solid phase; or a step of freeze-drying an aqueous solution of the neutral HMO obtained after the UF step, NF step, active charcoal treatment, ion exchange treatment and/or chromatography on neutral solid phase; or a step of crystallizing the neutral HMO (provided the neutral HMO of interest exists in crystalline form) obtained after the UF step, NF step, active charcoal treatment, ion exchange treatment and/or chromatography on neutral solid phase. Alternatively, the neutral HMO obtained after the UF step, NF step, active charcoal treatment, ion exchange treatment and/or chromatography on neutral solid phase may be provided in a form of a concentrated aqueous solution or syrup by removing water, e.g. by means of distillation, preferably vacuum distillation, or nanofiltration.

When the neutral HMO of interest is isolated in spray-dried form, it is preferably conducted as disclosed in e.g. WO 2013/185780, or the spray-drying process is performed e.g. with the 15-65 w/v % aqueous solution of the neutral HMO of interest at a nozzle temperature of 110-190° C. and at an outlet temperature of 60-110° C., provided that the nozzle temperature is at least 10° C. higher than the outlet temperature.

When the neutral HMO of interest is isolated in crystalline form, the crystallization is preferably performed
from aqueous acetic acid as disclosed in WO 2016/095924 to isolate 2′-FL polymorph II according to WO 2011/150939, or
from aqueous methanol as disclosed in WO 2011/100980 to isolate the LNnT polymorph characterized therein.

### 3. Preferred Embodiments of the Invention

The embodiments of the invention, including the preferred and the more preferred embodiments, do not comprise electrodialysis step.

One embodiment of the method relates to obtaining or isolating a neutral HMO from the fermentation broth in which it has been produced, comprising the steps of:

a) setting the pH of said reaction broth to pH=3-5,
b) optionally cooling the pH-adjusted broth obtained in step a) to 5-15° C. and stored for 1-15 days,
c) heating the broth of step a) or step b) to 60-65° C.,
d) contacting the broth obtained in step c) to an ultrafiltration (UF) membrane with a molecular weight cut-off (MWCO) of around 5-1000 kDa, such as 10-1000 kDa, wherein the membrane is a ceramic membrane, optionally at 40-65° C.,
e) optional sterile filtration of the permeate obtained in step d),
f) contacting the permeate obtained in step d) or the sterile filtered permeate obtained in step e) with a nanofiltration (NF) membrane having a MWCO of 600-3500 Da, wherein the active (top) layer of the membrane is composed of polyamide, and wherein the MgSO₄ rejection factor on said membrane is around 20-90%, preferably 50-90%,
g) treating the retentate obtained in step f) with a strong cationic ion exchange resin in H⁺-form followed by a weak basic ion exchange resin in base form to demineralize the retentate,
h) active charcoal chromatography of the solution obtained in step g) at 30-60° C., wherein the amount of the charcoal is around 2-10 weight % of the neutral HMO contained in the load,
i) nanofiltration of the solution obtained in step h) with a membrane of 150-500 Da to concentrate the solution,
j) water removal by spray-drying the solution of step i) to obtain the neutral HMO as an amorphous solid or crystallizing the neutral HMO from the solution obtained in step i).

One embodiment of the method relates to obtaining or isolating a neutral HMO from the fermentation broth in which it has been produced, comprising the steps of:

a) setting the pH of said reaction broth to a pH of around 5.5,
b) centrifuging the broth obtained in step a),
c) optionally heating the supernatant of step b) to 60-65° C.,
d) contacting the supernatant of step b) or step c) to an ultrafiltration (UF) membrane with a molecular weight cut-off (MWCO) of around 5-1000 kDa, optionally at 40-65° C.,
e) contacting the permeate obtained in step d) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   f) active charcoal chromatography of the retentate obtained in step e) at 30-60° C., wherein the amount of the charcoal is around 2-10 weight % of the neutral HMO contained in the load,
   g) treating the solution obtained in step f) with a strong cationic ion exchange resin in H⁺-form followed by a weak basic ion exchange resin in base form to demineralize the solution,
   h) nanofiltration of the solution obtained in step g) with a membrane of 150-500 Da to concentrate the solution,
   i) spray-drying the solution of step i) to obtain the neutral HMO as an amorphous solid or crystallizing the neutral HMO from the solution obtained in step h).

One embodiment of the method relates to obtaining or isolating 2′-FL from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) active charcoal chromatography of the retentate obtained in step c) at 30-60° C., e) evaporating the solution obtained in step d) until the concentration of 2′-FL reaches around 400-700 g/l,
   f) crystallizing the 2′-FL from the solution obtained in step e) with the aid of methanol or acetic acid, preferably acetic acid, to obtain 2′-FL polymorph II.

One embodiment of the method relates to obtaining or isolating 2′-FL from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) active charcoal chromatography of the retentate obtained in step c) at 30-60° C.,
   e) contacting the solution obtained in step d) with a nanofiltration (NF) membrane having a MWCO of 600-3500 Da, wherein the active (top) layer of the membrane is composed of polyamide, and wherein the MgSO₄ rejection factor on said membrane is around 20-90%, preferably 50-90%,
   f) evaporating the permeate obtained in step e) or nanofiltering the permeate obtained in step e) with a membrane of 150-300 Da to concentrate the solution until the concentration of 2′-FL reaches around 400-700 g/l,
   g) crystallizing the 2′-FL from the solution obtained in step f) with the aid of acetic acid to obtain 2′-FL polymorph II.

One embodiment of the method relates to obtaining or isolating 2′-FL from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) treating the retentate obtained in step c) with a strong cationic ion exchange resin in H⁺-form followed by a weak basic ion exchange resin in base form to demineralize the solution,
   e) active charcoal chromatography of the solution obtained in step d) at 30-60° C.,
   f) evaporating the solution obtained in step e) or nanofiltering the solution obtained in step e) with a membrane of 150-300 Da to concentrate the solution,
   g) crystallizing the 2′-FL from the solution obtained in step f) with the aid of acetic acid to obtain 2′-FL polymorph II or spray-drying the solution obtained in step f) to obtain 2′-FL as an amorphous powder.

One embodiment of the method relates to obtaining or isolating a neutral HMO from the fermentation broth in which it has been produced, comprising the steps of:

a) setting the pH of said reaction broth to a pH of around 3-6,
b) optionally heating the broth of step a) to 30-65° C.,
c) contacting the broth of step a) or step b) to an ultrafiltration (UF) membrane with a molecular weight cut-off (MWCO) of around 10-1000 kDa, optionally at 40-65° C.,
d) contacting the permeate obtained in step c) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   e) active charcoal chromatography of the retentate obtained in step d) at 30-60° C., wherein the amount of the charcoal is around 2-10 weight % of the neutral HMO contained in the load,
   f) treating the solution obtained in step e) with a strong cationic ion exchange resin in H⁺-form followed by a weak basic ion exchange resin in base form to demineralize the solution,
   g) nanofiltration of the solution obtained in step f) with a membrane of 150-300 Da to concentrate the solution,
   h) crystallizing the neutral HMO from the solution obtained in step g), wherein the neutral HMO is 2′-FL, LNnT or LNT.

One embodiment of the method relates to obtaining or isolating 3-FL from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) treating the retentate obtained in step c) with a strong cationic ion exchange resin in H⁺-form followed by a weak basic ion exchange resin in base form to demineralize the solution,
   e) active charcoal chromatography of the solution obtained in step d) at 30-60° C.,
   f) evaporating the solution obtained in step e) or nanofiltering the solution obtained in step e) with a membrane of 150-300 Da to concentrate the solution,
   g) spray-drying the solution obtained in step f) to obtain 3-FL as an amorphous powder.

One embodiment of the method relates to obtaining or isolating LNnT from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane having a MWCO of 600-3500 Da, wherein the active (top) layer of the membrane is composed of polyamide, and wherein the MgSO₄ rejection factor on said membrane is around 20-90%, preferably 50-90%,
d) active charcoal chromatography of the retentate obtained in step c) at 30-60° C.,
e) treating the solution obtained in step d) with a strong cationic ion exchange resin in H⁺-form followed by a weak basic ion exchange resin in base form to demineralize the solution,
f) evaporating the solution obtained in step e) or nanofiltration of the solution obtained in step e) with a membrane of 150-300 Da to concentrate the solution,
g) crystallizing the LNnT from the solution obtained in step f) with the aid of methanol.

One embodiment of the method relates to obtaining or isolating LNnT from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane having a MWCO of 600-3500 Da, wherein the active (top) layer of the membrane is composed of polyamide, and wherein the MgSO₄ rejection factor on said membrane is around 20-90%, preferably 50-90%,
d) treating the retentate obtained in step c) with a strong cationic ion exchange resin in H⁺-form followed by a weak basic ion exchange resin in base form to demineralize the solution,
e) active charcoal chromatography of the solution obtained in step d) at 30-60° C.,
f) evaporating the solution obtained in step e) or nanofiltration of the solution obtained in step e) with a membrane of 150-300 Da to concentrate the solution,
g) crystallizing the LNnT from the solution obtained in step f) with the aid of methanol.

One embodiment of the method relates to obtaining or isolating LNnT from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) active charcoal chromatography of the retentate obtained in step c) at 30-60° C.,
   e) treating the solution obtained in step d) with a strong cationic ion exchange resin in H⁺-form followed by a weak basic ion exchange resin in base form to demineralize the solution,
   f) chromatography of the solution obtained in step e) on a bromine functionalized polystyrene cross-linked with divinylbenzene (PS-DVB) hydrophobic stationary phase and to collect the fractions enriched in LNnT,
   g) evaporating the fractions obtained in step f) or nanofiltration of the fractions obtained in step f) with a membrane of 150-300 Da to concentrate the solution,
   h) spray-dying the solution obtained in step g) to obtain LNnT as amorphous powder.

One embodiment of the method relates to obtaining or isolating LNnT from the fermentation broth in which it has been produced, comprising the steps of:

a) setting the pH of the broth to a pH from around 3 to around 6, and/or warming up said broth to a temperature of about 35-65° C.,
b) contacting the broth of step a) to an ultrafiltration (UF) membrane with a molecular weight cut-off (MWCO) of around 5-1000 kDa, optionally at 40-65° C.,
c) contacting the permeate obtained in step b) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,
   d) treating the retentate obtained in step c) with a strong cationic ion exchange resin in H⁺-form followed by a weak basic ion exchange resin in base form to demineralize the solution,
   e) active charcoal chromatography of the solution obtained in step d) at 30-60° C.,
   f) chromatography of the solution obtained in step e) on a bromine functionalized polystyrene cross-linked with divinylbenzene (PS-DVB) hydrophobic stationary phase and to collect the fractions enriched in LNnT,
   g) evaporating the fractions obtained in step f) or nanofiltration of the fractions obtained in step f) with a membrane of 150-300 Da to concentrate the solution,
   h) spray-dying the solution obtained in step g) to obtain LNnT as amorphous powder.

### EXAMPLES

### General:

The concentration of the neutral HMO of interest was assayed by HPLC on TSKgel Amide-80 (150 mm×4.6 mm, particle size: 3 μm) with 64 v/v % acetonitrile at flow rate of 1.1 ml/min and at 25° C. using refractive index detector at 37° C.

The concentration of organic impurities was measured by HPLC on apHera NH₂ polymer (250 mm×4.6 mm; 5 μm) with 72 v/v % acetonitrile at flow rate of 1.1 ml/min and 25° C. using charged aerosol detector (CAD).

### Example 1

Fermentation: 2′-FL-containing broth was generated by fermentation using a genetically modified E. coli strain of LacZ⁻, LacY⁺ phenotype, wherein said strain comprises a recombinant gene encoding an α1,2-fucosyl transferase enzyme which is able to transfer fucose of GDP-fucose to the internalized lactose and genes encoding a biosynthetic pathway to GDP-fucose.

The fermentation was performed by culturing the strain in the presence of exogenously added lactose and a suitable carbon source, e.g. according to WO 2015/197082, thereby producing 2′-FL which was accompanied with DFL and unreacted lactose as major carbohydrate impurities in the fermentation broth.

After the fermentation was completed, the fermentation broth was subjected to cooling to 10° C. and 25% sulfuric acid solution was added during several hours until pH stabilized at 4.0.

The obtained broth (16.69 kg) contained 99.86 g/l of 2′-FL, 11.4 g/l of lactose and 3.61 g/l of DFL with a conductivity of 5.65 mS/cm and BWM (bio-wet mass) of 28.3%, which is calculated as a wet solid mass after removal of supernatant obtained by centrifugation of a small broth sample at room temperature divided by the initial mass of that broth sample.

A part of the broth (cca. 10 kg) was transferred to MMS SW18 membrane filtration system equipped with a 15 kDa ceramic membrane and equilibrated at 60° C. for approximately 45 minutes. A batch ultrafiltration was initiated at TMP=6 bar and 0.9 m/s cross-flow velocity. The remaining broth was added in small portions. After the feed volume was reduced to approximately one half (concentration factor, CF=2), diafiltration (DF) was started with the same amount of water as the initial feed amount (16.7 kg, “1 volume DF”), which was added continuously at 5.4 l/h flow rate matching approximately the permeate flow-rate before the start of DF. As a result, 28.45 kg of UF permeate as a clear brown solution was obtained containing 52.8 g/l of 2′-FL, 4.94 g/l of lactose, 1.33 g/l of DFL and 26.3 mg/l of proteins with a Brix of 8.5, a conductivity of 5.96 mS/cm and pH of 4.43. Recovery yield of 2′-FL yield was 96%.

The obtained UF permeate (28.42 kg) was transferred in portions to another SW18 membrane filtration system equipped with spiral-wound Trisep-UA60 1812 membrane (MWCO 1000-3500 Da according to specifications). The NF permeate collection was initiated at TMP=35 bar until 20.38 kg of NF permeate was collected with a Brix of 0.9. The pH of the retentate was adjusted from 4.3 to 5.5 with 10 ml of 50% NaOH solution. At this point, continuous DF with 25 kg of water was initiated at 5.5 l/h flow rate. During the DF, the TMP was increased to 40.0 bar and temperature stabilized at 45° C. The so-obtained (1^{st}) NF retentate (5.76 kg, Brix 27.7, d=1.12 g/cm³, conductivity 3.02 mS/cm, pH 5.64) contained 202.43 g/l of 2′-FL, 7.24 g/l of lactose, 7.63 g/l of DFL and 100.1 mg/l of proteins, where lactose/2′-FL ratio was reduced from 9.4% in the initial UF permeate to 3.6%. The amounts of other small molecules were also efficiently reduced. Also, the overall 2′-FL purity in a solid sample after freeze-drying was improved from 62.4% (in UF permeate) to 82.6% (NF retentate). Calculated step yield was 78.6%. In order to improve the yield, the DF permeate (27.30 kg) was re-processed in the same NF system by concentration to a small volume (ca. 2.3 l) as described above followed by diafiltration with 10 kg of water to give a 2^{nd} NF retentate (1.365 kg). Combined recovery yield of 2′-FL in the 1^{st} and the 2^{nd} NF retentate was 94%. The 2′-FL recovery yield of the UF+NF steps was 75.4% (without reprocessing the 1^{st} NF), which was improved to 90% by reprocessing the 1^{st} NF permeate.

The 1^{st} NF retentate (5.74 kg) was passed through a 1-litre column with tube internal diameter of 5 cm packed with 0.8 l of regenerated Dowex 88 resin (strong cation exchanger with sulfonic acid groups, H⁺-form) connected to a second 1-litter column packed with regenerated 0.8 l of Dowex 66 resin (weak anion exchanger, tertiary amine, free-base form) at approximately 2.4 l/h flow rate. The initial void volume of 640 ml was discarded followed by collection of the main 20 fraction. After the entire feed solution was consumed, the elution with ca. 1.6 l of deionized water was continued with the same flow-rate. The collected main fraction weighted 6760 g (density 1.09, Brix 21.8, conductivity 0.152 mS/cm, pH 6.21) and contained 180.17 g/l of 2′-FL, 5.97 g/l of lactose, 6.2 g/l of DFL and only 0.9 mg/l of proteins with near quantitative 2′-FL recovery. In addition, more than 95% reduction in salt content was estimated from the conductivity drop, the colour was also substantially reduced from dark brown to pale yellow. In another run with similar feed amount and composition, the colour reduction was quantified by UV-light adsorption at 400 nm from >3.0 in the NF retentate to 0.44 after de-mineralization. To illustrate the efficacy, the load on the resins was around 2 kg/l wet resin for the total solids and around 1.4 kg/l wet resin for 2′-FL. If the NF step was performed with an NF membranes of 150-300 kDa MWCO, at least 5 times more resin (meaning not more than ⅕^{th} load) is necessary to achieve the same low colour and conductivity.

The pale-yellow solution (6.74 kg) obtained in the above demineralization step was passed through granulated active charcoal (88 g, 6 w/w % relative to total solids in the feed) suspended in water and packed in a heat-jacketed GE XK-16/100 column at 60° C. and at 1.5 l/h flow rate followed by 200 ml of deionized water to provide a colourless clear solution (6919 g, density 1.086, Brix 20.7, conductivity 0.16 mS/cm, pH 6.1) containing 174.65 g/l of 2′-FL, 5.60 g/l of lactose, 5.95 g/l of DFL, and <1 mg/l of proteins with near quantitative 2′-FL yield. In another run with similar feed amount and composition, colour reduction was quantified by UV-light adsorption at 400 nm to give a substantial reduction from 0.44 in the feed to 0.01 after charcoal chromatography.

The solution from the previous step was concentrated under reduced pressure to 3.45 kg (Brix 41.6) and the pH of the solution was adjusted with small amount of 37% HCl solution from pH=6.5 to 4.83. The obtained concentrated syrup was sterile filtered with 200 nm filter and washed with deionized water. The so-obtained syrup (3.97 kg, Brix 36.2) was freeze-dried in 7 portions to give 1405 g of final product which was transferred and milled into a white powder containing 86.07% of 2′-FL, 3.83% of Lactose, 4.75% of DFL and 2.81% of residual water; proteins <0.0017%, potassium <10 mg/kg, magnesium <5 mg/kg, sodium 220 mg/kg, copper <0.1 mg/kg, iron <0.5 mg/kg, manganese <0.1 mg/kg, lead <0.01 mg/kg, zinc 0.2 mg/kg, ammonium <50 mg/kg, phosphate <50 mg/kg, sulphate 20 mg/kg, chloride 282 mg/kg, sulphated ash 0.09%, endotoxin 0.003 EU/mg, total plate count (microbiological parameter)<10 cfu/g.

### Example 2

Several samples of 2′-FL fermentation broth were subjected to pH adjustment by addition of 25% H₂SO₄ solution under stirring at room temperature; pH was measured after equilibration for 20 min and after 2 hours. Samples of the obtained liquids were centrifuged at room temperature and other samples were thermostated at 60° C. for 10 min before centrifugation at room temperature. The obtained supernatants were analysed by Bradford test for soluble protein content. Results are summarized in a table below. As a result, combination of low pH (<4) and a moderate heat treatment (60° C.) can reduce the amount of soluble proteins up to 0.1 parts relative to the untreated broth.

| | | proteins [mg/l] in |
|---|---|---|
| pH after adjustment and | proteins [mg/l] in | supernatant after |
| equilibration for 20 min | supernatant at r.t. | 60° C. treatment |
| 6.3 (no adjustment) | 3561 | 3719 |
| 5.0 | 2777 | 2265 |
| 4.5 | 2213 | 1320 |
| 4.2 | 1839 | 1135 |
| 3.9 | 1574 | 808 |
| 3.3 | 900 | 390 |

### Example 3

Several 2′-FL fermentation broths generated on 20 l scale as described in Example 1 were adjusted to different pH values and processed under the same UF conditions at 60° C. as described in Example 1. The protein content in UF permeate was reduced 5 times at pH=3.8 compared to the UF run at pH=6.3 without pH-adjustment, which is in agreement with protein reduction in supernatant at similar pH values as given in Example 2. Moreover, practically no difference in protein content was observed at pH 5.8 and 6.3, however a 2-fold protein reduction could be achieved at pH 3.8 vs pH 4.35.

### Example 4

LNnT was made by fermentation using a genetically modified E. coli cell as disclosed in WO 2017/182965, thereby producing a broth containing LNnT which was accompanied by intermediate lacto-N-triose II, pLNnH and unreacted lactose.

The obtained broth was adjusted to pH=5.0 and an divided into three equal portions Each portion was processed by UF in batch mode with a 50 nm ceramic membrane at 40° C., 50° C. and 60° C., respectively, in the same way as described for 2′-FL in Example 1 including concentration to CF=2.0 followed by DF with 1 volume of water relative to initial feed amount. LNnT yields and protein content in the UF permeate are summarized in the table below.

| T(° C.) | LNnT yield | proteins, mg/l |
|---|---|---|
| 40 | 53% | 141 |
| 50 | 65% | 615 |
| 60 | 88% | 672 |

1. A method for obtaining or isolating a neutral human milk oligosaccharide (HMO) from the reaction milieu in which it has been produced, comprising the steps of:
i) optionally, centrifugation, microfiltration of the reaction milieu, or filtration of the reaction milieu on a filter press or a drum filter to obtain a pre-treated reaction milieu,
ii) setting the pH of the pre-treated reaction milieu from step i) or the reaction milieu directly to 3-6, and
iii) contacting the reaction milieu obtained in step ii) to an ultrafiltration (UF) membrane with a molecular weight cut-off (MWCO) of 5-1000 kDa and collecting the permeate,
with the proviso that when step i) is not carried out, then the UF membrane is a non-polymeric membrane.

2. The method of claim 1, wherein molecular weight cut-off of the UF membrane of step iii) is 10-1000 kDa.

3. The method of claim 1, wherein step ii) comprises setting the pH of and warming up the pre-treated reaction milieu from step i) or the reaction milieu directly.

4. The method of claim 1, wherein step i) is not carried out and wherein the non-polymeric membrane of step iii) is a ceramic membrane.

5. The method of claim 1, wherein step iii) is carried out at 45-65° C.

6. The method of claim 5, wherein the UF membrane in step iii) is a ceramic membrane, and the method further comprises the step of contacting the permeate obtained in step iii) with a nanofiltration (NF) membrane and collecting the retentate.

7. The method of claim 6, wherein the NF membrane has a molecular weight cut-off (MWCO) of 600-3500 Da, the active (top) layer of the NF membrane is composed of polyamide, and wherein the MgSO₄ rejection factor on said membrane is around 50-90%.

8. The method of claim 6, wherein the nanofiltration retentate is treated with an ion exchange resin, wherein the treatment with ion exchange resin consists of the treatment of the NF retentate with a strong cation exchange resin in H⁺-form directly followed by a treatment with a weak anion exchange resin in free base form.

9. The method of claim 6, wherein the nanofiltration retentate is treated with active charcoal, at 30-60° C., wherein the amount of the charcoal is around 2-10 weight % of the neutral HMO contained in the nanofiltration retentate, to give an active charcoal eluate.

10. The method of claim 8, wherein the eluate of the ion exchange treatment is treated with active charcoal, at 30-60° C., wherein the amount of the charcoal is around 2-10 weight % of the neutral HMO contained in the eluate, to give an active charcoal eluate.

11. The method of claim 9, wherein active charcoal eluate is treated with an ion exchange resin, wherein the treatment with ion exchange resin consists of the treatment of the active charcoal eluate with a strong cation exchange resin in H⁺-form directly followed by a treatment with a weak anion exchange resin in free base form.

12. The method of claim 1, wherein the neutral HMO is 2′-FL, 3-FL, DFL, LNT, LNnT or LNFP-I.

13. The method of claim 12, wherein the neutral HMO is 2′-FL or LNnT.

14. The method of claim 1, wherein the reaction milieu in which the neutral HMO has been produced is a fermentation broth.

15. The method of claim 14, wherein the fermentation is performed by a genetically modified microorganism.

16. The method of claim 15, wherein the genetically modified microorganism is E. coli cell of LacY⁺ phenotype or LacZ⁻, LacY⁺ phenotype.

17. The method of claim 15, wherein the genetically modified microorganism is E. coli.

18. The method of claim 1, wherein step ii) further comprises warming up the pre-treated reaction milieu from step i) or the reaction milieu directly to 35-65° C.

19. The method of claim 9, wherein the nanofiltration retentate is subjected to chromatography on active charcoal.

20. The method of claim 10, wherein the eluate is subjected to chromatography on active charcoal.
